(19) United States
(12) Patent Application Publication
(10) Pub. No.: US 2023/0372439 A1
(43) Pub. Date: Nov. 23, 2023

(54) USE OF THE SESTRIN2 FOR THE TREATMENT OF CONTRAST-INDUCED ACUTE KIDNEY INJURY

(71) Applicant: UIF (University Industry Foundation), Yonsei University, Seoul (KR)

(72) Inventors: Yong Eun CHUNG, Seoul (KR); Hye Won OH, Seoul (KR)

(21) Appl. No.: 18/199,012

(22) Filed: May 18, 2023

(30) Foreign Application Priority Data
May 20, 2022 (KR) 10-2022-0061950

(51) Int. Cl. A61K 38/17; A61P 13/12

(52) U.S. Cl. CPC A61K 38/1709; A61P 13/12

(57) ABSTRACT

The present invention relates to a use of sestrin2 for preventing or treating acute kidney injury. According to the present invention, sestrin2 regulates oxidative stress to attenuate mitochondrial damage and cell death, alleviate the damage to renal cells, and reduce the expression of kidney injury markers, and thus can be utilized as a target for the prevention or treatment of acute kidney injury.

### CROSS-REFERENCE TO RELATED APPLICATION

This application claims priority to and the benefit of Korean Patent Application No. 10-2022-0061950, filed on May 20, 2022, the disclosure of which is incorporated herein by reference in its entirety.

### BACKGROUND

### 1. Field of the Invention

The present invention relates to a use of sestrin2 for preventing or treating acute kidney injury.

### 2. Discussion of Related Art

Acute kidney injury (AKI) or acute renal failure refers to a clinical syndrome that causes rapid deterioration of renal function and is caused by various causes such as reduction in renal blood flow, glomerulonephritis, use of nephrotoxic antibiotics and anticancer drugs. Acute kidney injury is accompanied by deterioration in glomerular filtration rate (GFR), decrease in urine output, azotemia due to accumulation of nitrogenous waste products, a fluid and electrolyte imbalance, and the like. Such acute kidney injury is very common in hospitalized patients and significantly increases patient morbidity and mortality, and increases the risk of developing chronic kidney disease (CKD) and end stage renal disease (ESRD).

Acute kidney injury has been reported to occur in 5 to 10% of all hospitalized patients and up to 60% of intensive care unit patients. In addition, the incidence of acute renal injury is increasing, leading to an increase in the incidence of end stage renal failure.

The causes of acute kidney injury can be broadly divided into three categories based on the kidneys, and can be classified into prerenal kidney injury caused by an obstruction of blood flow to the kidneys, renal kidney injury, which is a problem of the kidneys themselves, and postrenal kidney injury caused by an obstruction of a portion of the urinary tract from the tubule to the urethra. Prerenal kidney injury occurs when the volume of blood supplied to the kidneys decreases, and may occur when renal blood flow decreases due to severe dehydration caused by vomiting or diarrhea, heart failure, liver cirrhosis, sepsis, and the like. Renal kidney injury may occur due to the occurrence of a glomerular disease, tubular disease, epileptic disease, renal vascular disease, and the like in the kidneys themselves. Postrenal kidney injury may occur when there is a problem with the passage of urine, and it may occur when the urinary tract is blocked by urinary stones or tumors, and the like.

As described above, acute kidney injury may occur due to various causes, but is not necessarily induced by one factor, and kidney injury may occur due to the combined action of various causes or the interaction of other causes.

Acute kidney injury caused by a contrast medium may occur following the administration of a contrast medium used in medical imaging procedures such as computed tomography (CT) scans and angiographies, and is called contrast-induced acute kidney injury (CI-AKI). In particular, the exact mechanism of acute kidney injury caused by a contrast medium is unknown to date, but it is known that when iodine fixed to the benzene ring of a contrast medium becomes free iodine ions, it causes direct damage to renal tubular cells or vascular endothelial cells in the kidneys. Such damage reduces nitric oxide (NO) that dilates blood vessels, and increases intracellular reactive oxygen (oxidative stress) to promote vasoconstriction, which causes ischemic injury to the renal parenchyma, and consequently, cell death, and it is speculated that contrast-induced nephropathy occurs through this series of processes. Due to this series of processes, reactive oxygen is known to be the main cause of acute kidney injury caused by a contrast medium (hospital-acquired acute kidney injury).

The incidence of contrast-induced acute kidney injury may vary depending on patient factors, the type of procedure performed, the administered contrast route and the definitions applied. Specifically, it has been reported that the incidence of contrast-induced acute kidney injury is estimated to be between 1% and 2% in patients with normal renal function, but the frequency of incidence increases to 12 to 27% in patients with pre-existing reduced renal function. It has been reported that the incidence increases up to 50%, particularly, in high-risk patients such as patients with dehydration, diabetic nephropathy, renal injury, volume depletion or congestive heart failure and elderly patients, and when contrast-induced acute kidney injury develops in such patients, the severity is also higher than in the general population. In addition, contrast-induced acute kidney injury accounts for about 12% of acute kidney injury cases occurring in hospitals, and also acts as one of the three leading causes of acute renal failure occurring in hospitalized patients, along with ischemic acute renal failure (42%) and acute renal failure due to urinary obstruction (18%).

Despite these risks, there are still no therapeutic agents that directly target kidney injury caused by a contrast medium, and the focus is on prevention, and for prevention, only measures, such as adequate hydration via a vein before and after the administration of a contrast medium, the use of the minimal amount of a contrast medium, a method of administering sodium bicarbonate serving to reduce the production of reactive oxygen by increasing the pH of the renal medulla and urine, and avoidance of contrast media in patients with risk factors for pre-existing renal disease or other types of renal injury, are taken. Furthermore, the number of elderly patients and patients vulnerable to contrast-induced kidney injury such as diabetes, hypertension and heart failure patients among target patients undergoing examinations using contrast media is continuously increasing. Therefore, there is an urgent need for the development of a drug for treating kidney injury caused by a contrast medium.

Thus, the present inventors completed the present invention by confirming that sestrin2 regulates oxidative stress in a mouse model to attenuate mitochondrial damage and cell death, thereby ameliorating kidney injury.

### SUMMARY OF THE INVENTION

An object of the present invention is to provide a composition for preventing or treating acute kidney injury, containing sestrin2 as an active ingredient.

In order to solve the above problem, the present inventors conducted drug screening to identify a material that has a function of inhibiting acute kidney injury, and as a result, identified the efficacy of sestrin2 in preventing or treating acute kidney injury caused by a contrast medium.

Thus, the present invention provides a novel use of sestrin2 for preventing or treating acute kidney injury caused by a contrast medium.

In the present invention, “sestrin2 (GenBank accession no. NP_113647.1)” refers to a protein encoded by the SESN2 gene located on human chromosome 1, and consists of a sequence of 480 amino acids. Sestrin2 is known as a protein involved in regulating cellular responses to stress such as oxidative stress and metabolic stress.

In the present invention, “acute kidney injury (AKI),” or “acute renal failure (ARF)” refers to a rapid deterioration in renal function over several hours to several days. Persistent acute kidney injury may induce fluid, electrolyte and acid-base imbalances and loss of ability to regulate hormones, and induce multiple organ failure, including central nervous system, immune and blood coagulation dysfunction, and may have a negative impact on patient prognosis. Acute kidney injury may be defined as the case where serum creatinine increases 0.3 mg/dL or more within 48 hours, serum creatinine increases 1.5-fold or more from the baseline value or the value within the previous week, or urine output is less than 0.5 ml/kg/h for 6 hours (Kidney Disease: Improving Global Outcomes (KDIGO)).

In an exemplary embodiment of the present invention, the acute kidney injury may be induced by oxidative stress. In the present invention, “oxidative stress” refers to tissue damage induced by relative overproduction of reactive oxygen species (ROS) due to an imbalance between reactive oxygen species (ROS) production and antioxidant defense mechanisms for biomolecules, cells, and tissues. Here, “reactive oxygen species” may be referred to as activated oxygen, reactive oxygen, or activated oxygen species and mean the same material.

The present inventors confirmed that sestrin2 alleviates acute kidney injury by regulating oxidative stress to attenuate mitochondrial damage and cell death. According to an exemplary embodiment of the present invention, both 8-OHdG and MDA, which are reactive oxygen markers, were significantly decreased when an acute kidney injury model was treated with sestrin2 (FIG. 3). In addition, according to an exemplary embodiment of the present invention, when an acute renal injury model was treated with sestrin2, cristae damage, size increases, and vacuolization in mitochondria were alleviated (FIGS. 4 to 5), reduced ATP synthesis levels were restored (FIG. 6), and cell death marker expression was reduced (FIG. 8).

In an exemplary embodiment of the present invention, the acute kidney injury may be induced by a contrast medium. The “contrast medium (CM)” of the present invention refers to a drug that increases the contrast of an image by artificially increasing the X-ray absorption difference of each tissue such that tissues or blood vessels can be seen in radiological examinations such as magnetic resonance imaging (MRI) and computed tomography (CT) by injecting the contrast medium into the stomach, intestinal tract, blood vessels, subarachnoid space, joint cavities, and the like. For example, the contrast medium of the present invention may be an iodine-containing contrast medium, a negative contrast medium such as barium sulfate, or a positive contrast medium such as air, gas, or carbon dioxide, may be preferably a positive contrast medium. For example, the contrast medium may be iohexol, iopromide, iopamidol, iomeprol, ioversol, iobitridol, or iodixanol, but is not limited thereto. Further, the contrast medium may cause kidney damage by generating free iodide ions.

In the present invention, the contrast-induced acute kidney injury may be defined as an acute kidney injury in which a blood creatinine value is increased 25% or more or 0.5 mg/dl or more within 24 to 72 hours after the use of the contrast medium compared to the existing value, instead of the reduction in renal function due to other causes such as hypotension, use of other nephrotoxic drugs, urinary tract obstructions and embolisms.

According to an exemplary embodiment of the present invention, when an acute kidney injury model was treated with sestrin2, damage to renal tubular cells was alleviated (FIG. 9), the expression of kidney injury markers was significantly reduced (FIG. 10), and serum creatinine (Scr) and blood urea nitrogen (BUN), which are renal function indicators, were significantly reduced (FIG. 11). In addition, it was confirmed that when sestrin2 was administered in a kidney injury model, the contrast medium excretion rate was restored compared to the control (FIG. 12). Therefore, sestrin2 may be usefully used for the treatment of a disease induced by increased oxidative stress, particularly, acute kidney injury.

The present invention also provides a pharmaceutical composition for preventing or treating acute kidney injury, containing sestrin2 as an active ingredient; a use of sestrin2 for preparing a pharmaceutical composition for preventing or treating acute kidney injury; and a method for preventing or treating acute kidney injury, the method including administering a therapeutically effective amount of sestrin-2 to a subject.

All the contents described regarding sestrin2 and the use thereof in treating acute kidney injury may be applied as is or mutatis mutandis to the composition.

As used herein, the term “prevention” refers to all actions that suppress or delay acute kidney injury by administration of the pharmaceutical composition according to the invention, and the term “treatment” refers to all actions that ameliorate or benefit the symptoms of acute kidney injury by administration of the pharmaceutical composition according to the present invention.

In the present invention, “subject” refers to a subject in need of prevention or treatment of a disease, and more specifically, it includes any mammal in need of prevention or treatment of acute kidney injury, such as not only a human and a primate, but also a domestic animal such as a cow, a pig, a sheep, a horse, a dog and a cat without limitation, but may be preferably a human.

For the administration route of the pharmaceutical composition, the drug can be administered through any general route as long as the drug can reach a target tissue, and can be administered to a subject orally or parenterally. For example, the administration routes may be intraperitoneal administration, intravenous administration, intraarterial administration, intramuscular administration, subcutaneous administration, intradermal administration, oral administration, topical administration (including skin application, drip administration and inhalation), transdermal administration, intradural and epidural administration, intranasal administration, intraocular administration, intrapulmonary administration, intrarectal administration, intravaginal administration, and the like, but is not limited thereto. In addition, the pharmaceutical composition may be administered in the form of any convenient pharmaceutical product, such as a tablet, a powder, a granule, a capsule, an oral liquid, a solution, a dispersion, a suspension, a syrup, a spray, a suppository, a gel, an emulsion, a patch, and the like. However, since the pharmaceutical composition may be digested upon oral administration, it may be desirable to coat an active agent or formulate the oral composition so as to protect it from degradation in the stomach.

The pharmaceutical composition of the present invention may additionally include a pharmaceutically or physiologically acceptable carrier, excipient and diluent. As used herein, the term “pharmaceutically acceptable carrier, excipient and diluent” refers to a carrier, excipient and diluent that do not stimulate an organism and do not inhibit the biological activity or properties of an administered compound. Examples of a suitable carrier, diluent, and excipient that may be included in such a composition include saline, sterile water, Ringer's solution, buffered saline, an albumin injection solution, glycerol, ethanol, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, hypromellose, polyvinylpyrrolidone, water, methylhydroxybenzoate, propylhydroxybenzoate, talc, magnesium stearate, mineral oil, and the like. When the composition is formulated, the composition may additionally include a typical dispersing agent, filler, extender, binder, disintegrating agent, surfactant, anti-aggregating agent, lubricant, wetting agent, fragrant, emulsifier, preservative, lyophilized formulation and the like.

In the present invention, the pharmaceutical composition may be administered in the form of an injection, although not limited thereto. The injection may be formulated as an intravenous or subcutaneous injection. In the case of a parenteral injection, the parenteral injection may contain ingredients included in a general injection composition. For example, the injection composition contains a liquid carrier such as sterile water, water for injection, and physiological saline. Additionally, an amino acid, sugar, a lipid, a vitamin, an electrolyte, a pH adjuster, a stabilizer, an osmotic pressure adjuster or a solubilizing adjuvant may be further contained.

In the present invention, when the pharmaceutical composition is topically administered, it may be formulated as an ointment, a gel, a cream, a lotion, and the like. The mode of topical administration is not limited thereto, but may be, for example, application to the skin, instillation into the eyes, transdermal permeation using microneedles, intradermal injection, and the like. For example, it may be desirable to apply the composition or attach a patch formulation containing the composition to the skin. The composition may include, for example, a base, an excipient, a lubricant and a preservative. For example, when administered in the form of a collyrium, the pharmaceutical composition may further contain a buffer, a viscosity agent, an isotonic agent, a pH adjuster, and a solvent. In addition, when administered in the form of an ointment for skin application, the pharmaceutical composition may further contain a gelling agent, a stabilizer, an emulsifier, and a suspending agent.

Furthermore, the pharmaceutical composition of the present invention may be applied differently depending on the purpose of administration and disease. The amount of active ingredient to be actually administered may be appropriately selected by those skilled in the art in consideration of various related factors, that is, a disease to be treated, the severity of the disease, co-administration with other drugs, drug activity, drug sensitivity, the age, sex, and body weight of a patient, diet, administration time, administration route and administration ratio of the composition. The composition may be administered once or in 1 to 3 divided doses a day, although the dosage and route of administration may be adjusted according to the type and severity of the disease.

In the present invention, the content of sestrin2 may be 10 to 150 μg/kg, 10 to 140 μg/kg, 15 to 130 μg/kg, 15 to 120 μg/kg, and 20 to 110 μg/kg, for example, 20 to 100 μg/kg, based on the total weight of the pharmaceutical composition, but is not limited thereto.

Furthermore, the pharmaceutical composition may be co-administered with a contrast medium. For example, the contrast medium may be iohexol, iopromide, iopamidol, iomeprol, ioversol, iobitridol, or iodixanol, but is not limited thereto.

The benefits and features of the present invention, and the methods of achieving the benefits and features will become apparent with reference to embodiments to be described below in detail. However, the present invention is not limited to the exemplary embodiments to be disclosed below, and may be implemented in various other forms, and the present exemplary embodiments are only provided for rendering the disclosure of the present invention complete and for fully representing the scope of the invention to a person with ordinary skill in the technical field to which the present invention pertains, and the present invention will be defined only by the scope of the claims.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 illustrates the sequence in which a recombinant adenovirus containing sestrin2 base sequence is inserted into an expression vector;

FIG. 2 illustrates the expression of sestrin2 protein in kidney tissue by quantification and qualification;

FIG. 3 is a set of graphs showing the reactive oxygen effect of recombinant adenovirus containing sestrin2;

FIG. 4 illustrates the observation of the degree of damage to mitochondria in kidney tissue cells in a kidney injury model caused by a contrast medium, using transmission electron microscopy;

FIG. 5 illustrates the comparison and analytical observation of the structural changes of mitochondria in each group using transmission electron microscopy;

FIG. 6 illustrates the ATP synthesis levels in homogenized kidney tissue;

FIG. 7 illustrates the quantitative analysis of the mRNA expression of pro-inflammatory markers (TNF-α, IL-6, IL-1α, and IL-β) in kidney tissue using real-time polymerase chain reaction;

FIG. 8 illustrates cell apoptosis-related markers (Bax, Bcl2, Cleaved caspase-3, and TUNEL) confirmed by immunohistochemistry and transmission electron microscopy (TEM);

FIG. 9 illustrates the results of microscopic observation of kidney tissue stained with hematoxylin and eosin (H&E) and periodic acid-Schiff (PAS);

FIG. 10 illustrates the comparison of the protein expression of kidney injury-related markers (KIM-1, Ngal, and IL-18) by an enzyme-linked immunosorbent (ELISA) method using urine and serum;

FIG. 11 illustrates a comparative analysis of serum creatinine (Scr) and blood urea nitrogen (BUN), which are diagnostic indicators of renal function, using serum; and

FIG. 12 illustrates a comparative analysis of the excretion rate of a contrast medium using images obtained by a Micro CT device for animals.

### DETAILED DESCRIPTION OF EXEMPLARY EMBODIMENTS

Experimental Methods

1) Kidney Injury Mouse Model Induced by Contrast Medium

In this study, using a control, a carrier (recombinant adenovirus containing sestrin2 protein), and a kidney injury model induced by a contrast medium using the mouse C57BL/6 lineage, the carrier (recombinant adenovirus containing sestrin2 protein) was administered to the contrast medium kidney injury model to divide the animals into 4 groups, and experiments were performed on 7 animals per group. To construct a kidney injury model induced by a contrast medium, drinking water was restricted 16 to 24 hours beforehand. Thereafter, NSAID-based kerotorolac tromethamine (Keromin, 1 mg/ml) which can induce deterioration of renal function and N-ω-Nitro-L-arginine methyl ester hydrochloride (L-NAME, 15 mg/kg) which reduces intracellular reactive oxygen scavenging were administered by intraperitoneal injection. 20 minutes later, an iopamidol 370 contrast medium (0.1 ml/kg) was administered by tail intravenous injection. 24 hours after model construction, molecular biological experiments were verified by sacrificing the mice to collect blood and tissues.
Group 1: Control: control
Group 2: Recombinant adenovirus containing sestrin2 (RS): administration of recombinant sestrin2
Group 3: Contrast-induced acute kidney injury (CI-AKI): acute kidney injury model due to contrast medium induction
Group 4: Contrast-induced acute kidney injury with recombinant adenovirus containing sestrin2 (CI-AKI+RS): RS administration to contrast agent-induced kidney injury model

2) Construction of Recombinant Adenovirus Containing Sestrin2 Protein

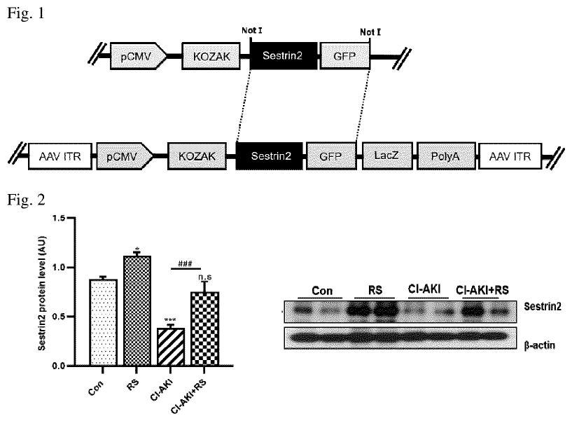

An amino acid protein containing a mouse sestrin base sequence was inserted into a pAAV-expression vector (FIG. 1). Through sequencing analysis, it was confirmed that the mouse sestrin2 base sequence was aligned in the vector. To increase the efficiency of transformation, DH5α competent cells were used in an LB agar medium to perform transformation with mouse sestrin2. The resulting colony was grown in the LB liquid, and a plasmid containing mouse sestrin2 in the expression vector was extracted using a Maxi-Prep kit.

3) Renal Function Assessment

Blood obtained after sacrificing mouse was centrifuged at 14000 rpm for 15 minutes to obtain serum. Serum creatinine and blood urea nitrogen were measured using a Cobas C502 apparatus.

4) Urine Collection

Urine was collected using a metabolic cage for 24 hours immediately prior to sacrifice. The collected urine was centrifuged at 14000 rpm and 4° C. for 20 minutes using a centrifuge. Only the supernatant was collected and used for analysis.

5) Enzyme-Linked Immunosorbent Assay (ELISA)

Mouse urine was used to verify kidney injury markers (Kim-1, Ngal, and IL-18). ELISA was performed with an ELISA kit according to the protocol, and the amount of materials in the sample was measured at a wavelength of 450 nm using a microplate reader.

6) Reactive Oxygen (Oxidative Stress) Experiment

A process of determining whether reactive oxygen was generated using 8-hydroxy-2′-deoxyguanosine (8-OHdG), which is a DNA damage marker in urine, and malondialdehyde (MDA) was performed according to a kit experimental method using serum and tissue. The amount of reactive oxygen generated in the sample was analyzed at a wavelength of 450 nm using a microplate reader.

7) ATP Synthesis Analysis

In order to verify the ATP synthesis level in kidney tissue, the tissue was crushed and homogenized using a homogenizer. Thereafter, the course of the experiment followed the method of the purchased reagents. Thereafter, the amount of ATP synthesized in the sample was analyzed at a wavelength of 570 nm using a microplate reader.

8) Real-Time Polymerase Chain Reaction

RNA was extracted using an RNA extraction kit using kidney tissue. Complementary DNA (cDNA) was synthesized using 1 μg of the extracted RNA. Synthesized cDNA and primers consisting of complementary sequences of a target to be amplified were mixed with the SYBR-Green reagent, and messenger RNA (mRNA) expression was quantitatively analyzed using QuantStudio3 real-time PCR equipment.

9) Western Blotting

In order to confirm protein expression in kidney tissue, the kidney tissue was homogenized with a protein extraction reagent, and an extracted protein was quantified using the Bradford method. Equal amounts of protein were loaded on an SDS-PAGE gel to perform separation according to a protein mass difference. Thereafter, the separated proteins were transferred to a PVDF membrane. Blocking was performed at room temperature for 1 hour using 3% bovine serum albumin (BSA) in order to prevent non-specific antibodies from binding to the protein transferred to the PVDF membrane. Thereafter, a primary sestrin2 antibody was diluted to 1:1000 and cultured at 4° C. for 16 to 24 hours. Thereafter, the PVDF membrane bound to the primary antibody was washed three to four times with Tris-buffered saline with 0.1% Tween (1×TBST) buffer for 20 minutes. Thereafter, an HRP-conjugated secondary antibody (goat anti-rabbit IgG-HRP) was diluted to 1:10000 and cultured at room temperature for 1 hour. Thereafter, the secondary antibody-bound membrane was washed three to four times using 1×TBST buffer for 20 minutes, an enhanced chemiluminescence (ECL) reagent for protein detection was exposed to the membrane, and then the expression level of protein was verified using an X-ray film.

10) Histopathology and Immunohistochemistry Analysis

Kidney tissue removed after sacrificing mice was fixed in 10% formalin at room temperature for 24 to 72 hours. For histopathological observation, the fixed tissue was made into paraffin blocks and cut into slices with a thickness of 4 μm. The sliced tissue was deparaffinized by exposure to xylene at different concentrations. Deparaffinized slices were stained using hematoxylin and eosin (H&E) and periodic acid-Schiff (PAS) reagents.

Deparaffinized blocks were sliced into a thickness of 4 μm for immunohistochemistry. Slides were exposed to methanol containing 0.3% hydrogen peroxide at room temperature for 10 minutes. The slides were blocked with 5% bovine serum albumin (BSA) to prevent binding of non-specific antibodies. Thereafter, primary antibodies (Bax, Bcl2, cleaved caspase-3) were diluted 1:100 using 5% BSA and allowed to react at room temperature for 1 hour. Thereafter, after washing three to four times with phosphate buffered saline (PBS), a secondary antibody (biotinylated goat anti-rabbit IgG-HRP) was diluted to 1:10000 with 5% BSA and allowed to react at room temperature for 1 hour. Stained slides were observed using a microscope.

11) TUNEL Assay Experiments

To verify cell death in the kidneys, experiments were performed according to the experimental method presented in the terminal deoxynucleotidyl transferase (TdT)-mediated dUTP nick-end-labeling (TUNEL) assay kit, and deparaffinized tissue slides were stained with a reagent and observed under a microscope.

12) Transmission Electron Microscopy

In order to observe structural changes in intracellular mitochondria, kidney tissue was fixed in a 0.1 M phosphate buffer containing 2% paraformaldehyde, 2% glutaraldehyde, and 0.5% calcium chloride (CaCl₂) for 3 to 4 days. The fixed sample was washed two to three times with a 0.1 M phosphate buffer for about 30 minutes. Thereafter, dehydrogenation was performed with ethanol at different concentrations of 50, 60, 70, 80, 90, and 100(%) for the dehydrogenation process. Thereafter, the sample was polymerized at 65° C. for 24 hours using a micro oven using a poly/Bed812 kit. The prepared blocks were sliced to a thickness of 80 nm using an ultramicrotome. Structures in cells and mitochondria were observed using a TEM (JEM-1011, JEOL, Tokyo, Japan) device.

13) Computed Tomography (CT)

Computed tomography was performed using a Quantum GX2 Micro CT imaging (Perkin Elmer, Waltham, MA, USA) device at the animal imaging center located within the research institute of the inventors. Micro CT images were measured using the following parameters. 1) tube current, 88 pA; 2) tube voltage, 90 kV; 3) scan time, 2 min; 4) field of view, 36 mm; 5) slice thickness=72 μm; and Cu 0.06+Al 0.5, X-ray filter). A CT contrast medium (0.2 ml, Visipaque, GE Healthcare, NJ, USA) was intravenously injected (iv) into the mouse tail for image acquisition. In the acquired images, the renal cortex and aorta of both kidneys were drawn as regions of interest (ROI), and the values were quantified.

### Example 1: Confirmation of Sestrin2 Expression in Contrast-Induced Acute Kidney Injury (CI-AKI) Mouse Model

Sestrin2 protein expression was verified in each of the four groups using a western blot.

It was confirmed that the expression of sestrin2 protein was statistically significantly decreased in a contrast-induced acute kidney injury mouse model (FIG. 2).

Further, as a result of administering recombinant adenovirus containing sestrin2 alone, it was shown that the expression of sestrin2 was significantly increased. As a result of administering the recombinant adenovirus containing sestrin2 protein in the contrast-induced acute kidney injury model, it was confirmed that the protein expression of sestrin2 was statistically significantly restored too. Results are expressed as mean±SEM for each group (n=7). Statistical significance: *P<0.05 Con vs RS and Con vs CI-AKI, ^{##}P<0.01 CI-AKI vs CI-AKI+RS (FIG. 2).

### Example 2: Confirmation of Occurrence of Intracellular Oxidative Stress and Verification of Effect of Recombinant Adenovirus Containing Sestrin2 in Contrast-Induced Acute Kidney Mouse Model

In order to confirm that reactive oxygen occurs and recombinant adenovirus containing sestrin2 decreases reactive oxygen in a contrast-induced acute kidney injury model, oxidative DNA damage (8-OHdG) values were confirmed in urine and malondialdehyde (MDA) values were confirmed in tissues and serum.

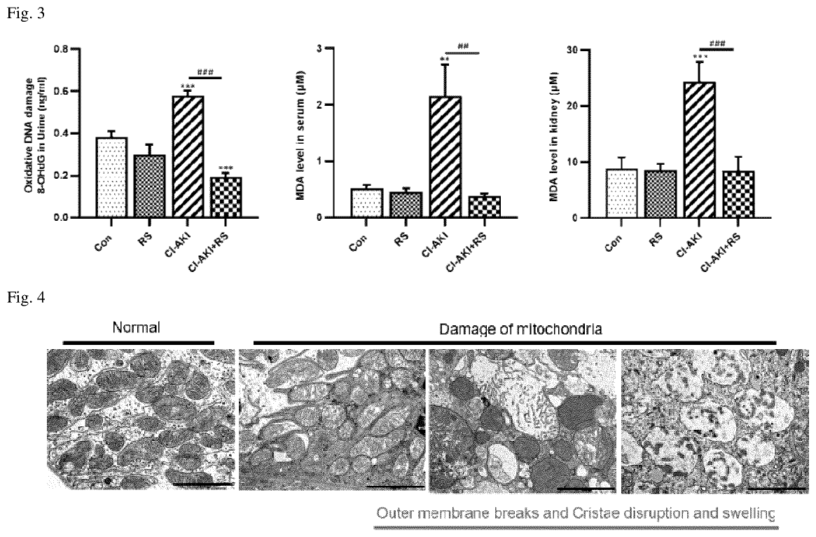

In the contrast-induced kidney injury model, two reactive oxygen markers were statistically significantly increased, and as a result of administering the recombinant sestrin2 protein, it was confirmed that reactive oxygen, which is the main mechanism of kidney injury, decreased. Results are expressed as mean±SEM for each group (n=7). For the results, statistics for each group (n=7) are expressed as mean±SEM. Statistical significance: ***P<0.05 Con vs CI-AKI and Con vs CI-AKI+RS, ^{##}P<0.01, ^{###}P<0.001 CI-AKI vs CI-AKI+RS (FIG. 3).

### Example 3: Structural Observation of Mitochondria in Contrast-Induced Acute Kidney Injury Mouse Model

It was confirmed, using transmission electron microscopy, that increased oxidative stress also affects cristae damage, an increase in mitochondrial size (swelling), and vacuolization, which decrease the efficiency of ATP synthesis in the mitochondrial structure. [magnification=50 k, scale bar=2000 nm] (FIG. 4).

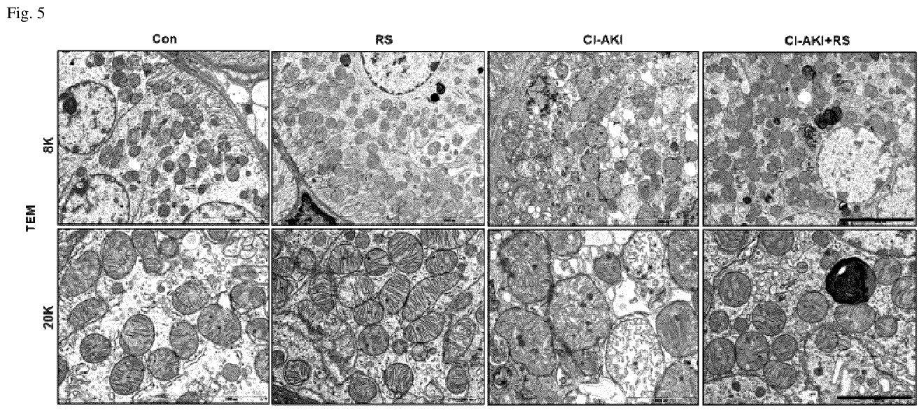

As a result of confirming the mitochondria of each of the four groups with TEM images, it was confirmed that cristae damage, an increase in size and vacuolization in mitochondria were alleviated as much as in the control. [magnification=8 k, scale bar=5000 nm] (FIG. 5).

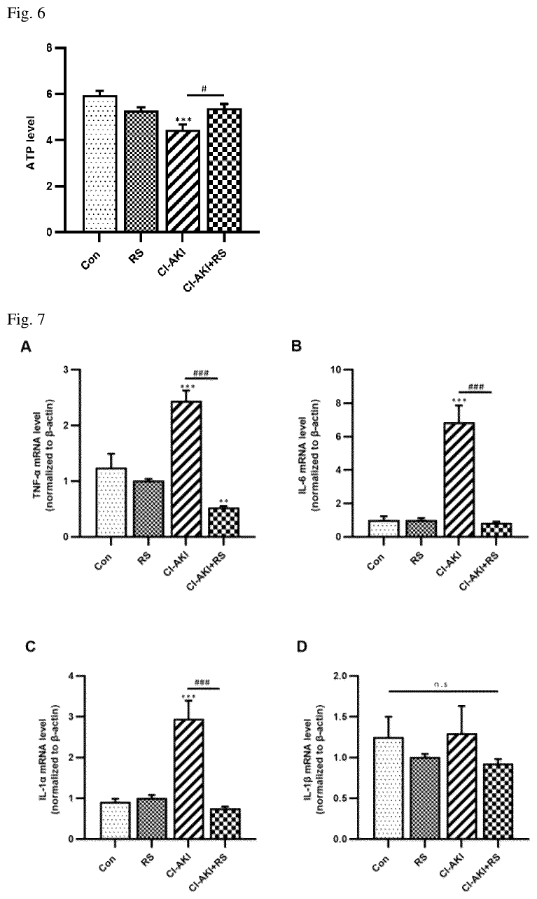

In addition, as a result of confirming the level of ATP synthesis in the renal tissue of each group, it could be confirmed that the synthesis of ATP was decreased in the contrast nephropathy model, but was restored by the administration of recombinant adenovirus containing sestrin2. Statistics are expressed as mean±SEM. Results are expressed as mean±SEM for each group (n=7). Statistical significance: ***P<0.001, Con vs CI-AKI, ^{#}P<0.05 CI-AKI vs CI-AKI+RS (FIG. 6).

### Example 4: Alleviation of Early Inflammatory Marker (Pro-Inflammatory) in Contrast-Induced Injury Mouse Model by Administration of Recombinant Adenovirus Containing Sestrin2

mRNA levels in tissue were confirmed using real-time polymerase chain reaction (PCR) analysis of TNF-α, IL-6, IL-1α, and IL-β, which are early inflammatory markers, in each of the four groups. As a result, it was verified that TNF-α, IL-6, and IL-1α, other than IL-1β, were statistically significantly increased in the contrast-induced kidney injury model, whereas the administration of recombinant adenovirus containing sestrin2 significantly decreased the early inflammatory values. For the results, statistics for each group (n=7) are expressed as mean±SEM. Statistical significance: ***P<0.001, Con vs CI-AKI, ^{###}P<0.05 CI-AKI vs CI-AKI+RS, n.s, no significance (FIG. 7).

### Example 5: Confirmation of Amelioration of Apoptosis in Contrast-Induced Kidney Injury Mouse Model by Administration of Recombinant Adenovirus Containing Sestrin2

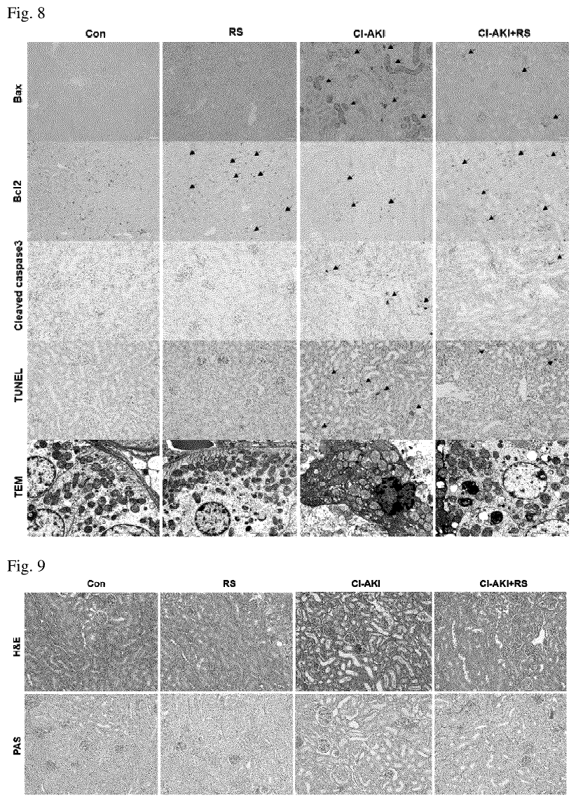

As a result of confirming the expression of cell death-related markers Bax, Bcl2, cleaved caspase 3 and TUNEL by immunostaining (Immunohistochemistry), it was confirmed that the stained dead cells were increased in the contrast-induced kidney injury model, and the administration of recombinant adenovirus containing sestrin2 significantly decreased the expression of dead cells. [magnification=20×, scale bar=50 μm, and arrows indicate apoptotic cells] (FIG. 8).

In addition, when cell death occurs, DNA condensation is observed in the nucleus, and it was confirmed that DNA condensation occurred due to cell death in the contrast-induced kidney injury model, and the kidney injury was alleviated by observing that cell death occurred less when recombinant adenovirus containing sestrin2 was administered using transmission electron microscopy [magnification=8 k, scale bar=5000 nm, and arrows indicate apoptotic cells]. (FIG. 8).

### Example 6: Verification of Histopathological Amelioration in Contrast-Induced Kidney Injury Mouse Model by Administration of Recombinant Adenovirus Containing Sestrin2

Histopathological verification was performed using hematoxylin and eosin (H&E) staining and periodic acid-Schiff (PAS) staining. It was observed that in the contrast-induced acute kidney injury model, the damage to renal tubular cells was exacerbated, and it was observed that in the results of administering recombinant adenovirus containing sestrin2, the damage to renal tubular cells was alleviated. [magnification=20×, scale bar=50 μm] (FIG. 9)

### Example 7: Verification of Protein Expression Renal Injury Markers Due to Recombinant Adenovirus Containing Sestrin2 in Contrast-Induced Acute Kidney Injury Mouse Model

Protein expression of KIM-1, NGAL, and IL-18 as representative markers for early diagnosis of kidney injury was verified using enzyme-linked immunosorbent assay (ELISA).

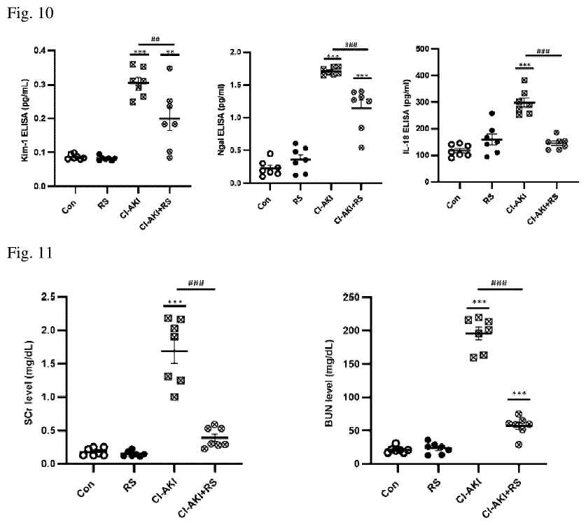

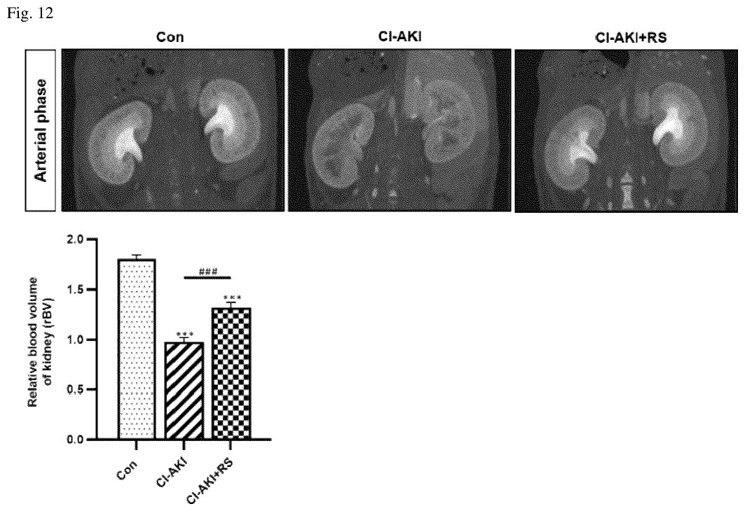

Kidney injury markers (KIM-1, NGAL, and IL-18) were statistically significantly increased in the contrast-induced acute kidney injury model. As a result of administering recombinant adenovirus containing sestrin2, it was verified that the expression of the kidney injury markers was significantly reduced. For the results, statistics for each group (n=7) are expressed as mean±SEM. Statistical significance: **P<0.01 and ***P<0.001, Con vs CI-AKI, Con vs CI-AKI+RS, ^{##}P<0.01 and ^{###}P<0.001 CI-AKI vs CI-AKI+RS (FIG. 10).

### Example 8: Verification of Effect of Recombinant Adenovirus Containing Sestrin2 in Contrast-Induced Acute Kidney Injury Mouse Model Through Renal Function Diagnostic Marker

Serum creatinine (Scr) and blood urea nitrogen (BUN), which are renal function indicators, were used for verification. Serum creatinine levels were increased 25% or more in the contrast-induced kidney injury model (defined as kidney injury caused by a contrast medium). In contrast, as a result of administering recombinant adenovirus containing sestrin2, it was shown that the levels of serum creatinine and blood urea nitrogen were statistically significantly decreased. Results are expressed as mean±SEM for each group (n=7). Statistical significance: ***P<0.001 Con vs CI-AKI, ^{###}P<0.001 CI-AKI vs CI-AKI+RS (FIG. 11).

### Example 9: Verification of Recombinant Adenovirus Containing Sestrin2 Effects with Computed Tomography (CT) Images in Contrast-Induced Acute Kidney Injury Mouse Model

A computed tomography (CT) device was used to verify how well the administered contrast medium was excreted through the urethra due to the administration of recombinant adenovirus containing sestrin2 to the kidneys injured by the contrast medium. A minimal amount of contrast medium was additionally administered to the tail vein of the mouse to acquire images. Although the excretion rate of the contrast medium was rapidly decreased in the contrast-induced kidney injury model, it was verified that the excretion rate was restored as a result of administering recombinant sestrin2. For the results, statistics for each group (n=5) are expressed as mean±SEM. Statistical significance: ***P<0.001, Con vs CI-AKI, Con vs CI-AKI+RS, ^{###}P<0.001 CI-AKI vs CI-AKI+RS.

According to the present invention, recombinant adenovirus containing sestrin2 attenuates oxidative stress, mitochondrial damage and cell death and thus can be utilized as a target for the prevention or treatment of acute kidney injury.

What is claimed is:

1. A pharmaceutical composition for preventing or treating acute kidney injury, containing sestrin2 as an active ingredient.

2. The pharmaceutical composition of claim 1, wherein the acute kidney injury is induced by a contrast medium.

3. The pharmaceutical composition of claim 2, wherein the contrast medium is iohexol, iopromide, iopamidol, iomeprol, ioversol, iobitridol, or iodixanol.

4. The pharmaceutical composition of claim 1, wherein the composition reduces intracellular oxidative stress.

5. The pharmaceutical composition of claim 1, further comprising a pharmaceutically acceptable carrier, excipient or diluent.

6. The pharmaceutical composition of claim 1, wherein a content of the sestrin2 is 20 μg/kg to 100 μg/kg based on a total weight of the composition.

7. A method for preventing or treating acute kidney injury, the method comprising administering the composition of claim 1 to a subject in need thereof.

8. The method of claim 7, wherein the acute kidney injury is induced by a contrast medium.

9. The method of claim 7, wherein the composition reduces intracellular oxidative stress.
